(19) United States
(12) Patent Application Publication
(10) Pub. No.: US 2004/0097543 A1
(43) Pub. Date: May 20, 2004

(54) Use of cyclic 2,4-diphosphoclycerates (cdgp) and/or derivatives thereof in cosmetic and dermatological formulations

(76) Inventor: Thomas Schwarz, Leichlingen (DE)

Correspondence Address: Robert Berliner, Fulbright & Jaworski, Twenty-Ninth Floor, 865 South Figueroa, Los Angeles, CA, 90017-2571, US

(21) Appl. No.: 10/344,969

(22) PCT Filed: Aug. 16, 2001
(86) PCT No.: PCT/EP01/09443

(30) Foreign Application Priority Data
Aug. 18, 2000 (DE) 100 40 931.8

(51) Int. Cl. A61K 31/46

(52) U.S. Cl. 514/304

(57) ABSTRACT

The invention relates to the inclusion of the low molecular weight substance (compatible solute), cyclic 2,4-diphosphoglycerate (cDGP), from extremophilic micro-organisms and/or derivatives of said compound, for example, an acid, a salt, or an ester, in cosmetic or dermatological formulations for protection of the skin from environmental influences and to increase the regeneration capacity. According to the invention, cyclic 2,4-diphosphoglycerate is implicated in active cell protection of the endogenous cell radical traps, antioxidants, the DNA, cell membranes and further cell compartments, by means of a protective effect of the above, against damaging environmental effects, for example, UV radiation, IR radiation, and environmental stress (thermal, chemical and physical). The compatible solute may serve as co-solvent and penetration improver for other active ingredients added to the cosmetic formulation, in order to not only stabilise the above in the cosmetic formulation, but also for active transport into deeper skin layers.

### TECHNICAL FIELD/SUBJECT OF THE INVENTION

Subject of the invention is the use of one or more chemical compounds selected from the compounds of formula Ia (cyclic diphosphoglycerate) in cosmetic and dermatological formulations. The invention, for example, relates to the manufacture of a cosmetic or dermatological formulation for the protection and stabilization of human skin cells as well as their organic constituents such as, for example, proteins, enzymes, membranes, nucleic acids or antioxidants.

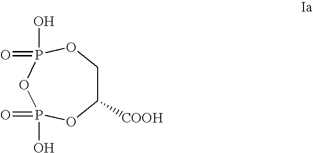

The invention embraces the use of physiologically compatible salts of the Ia and Ib compounds, the stereoisomeric forms of the compounds of formulas Ia and Ib and derivatives of these compounds (e.g. esters) for the use in cosmetic and dermatological formulations.

Since they originate from extremophilic microorganisms that by producing novel compatible solutes such as cyclic diphosphoglycerate protect themselves effectively against environmentally induced stress factors as UV radiation, heat, radicals, cold, dryness or osmotic or chemical stresses and, only in this way, are capable of surviving, these substances stand for a completely new approach to the protection of organisms and their components from harmful environmental influences.

The cell's inherent repair mechanism (DNA repair after UV damage, induction and stabilization of the chaperon proteins to newly form (partially) denaturized proteins and enzymes) can, for the first time, be stabilized and activated by cyclic diphosphoglycerate. Cyclic diphosphoglycerate, for the first time, enables the antioxidative potential of the cell (protein and DNA damage through free radicals, lipid oxidation of the cell membranes) to be maintained.

Due to their low molecular weight cyclic diphosphoglycerates penetrating through several skin layers enter the interior of the target cell and—acting as co-solvents or penetration intensifiers- are thus capable of carrying along other agents of cosmetic formulations (e.g. proteins, enzymes, vitamins, antioxidants) bringing them to the desired destination.

As a result of its chemical structure cyclic diphosphoglycerate is compatible with the majority of the customary cosmetic basic formulations and further adjustments to the desired and suitable form of application can be achieved by appropriately modifying the functional side chains. In this process and due to numerous hydroxy groups cyclic diphosphoglycerate features moisturizing properties and is very pleasant to the skin.

Compatible solutes act as natural solvents in extremophilic microorganisms and stabilize enzymes, proteins and other cell constituents by generating a more native, globular state that not only protects proteins against proteolysis but moreover keeps them in a more compact and active condition. As co-solvent or penetration intensifier for cosmetic agents cyclic diphosphoglycerate can be beneficially and broadly used for various applications particularly in view of a possible derivatization of individual functional groups.

Subject of the invention is thus the use of cyclic diphosphoglycerate as additive and agent offering a completely novel functioning principle in cosmetic and dermatological preparations and products.

### STATE OF THE ART

Sunscreens

Today's skin and sun-protective agents comprise two classes of UV blocking filters. Physically active UV blocking filters consist of inorganic compounds (e.g. titanium dioxide) that reflect the light rays before hitting the skin's surface. Chemically acting organic molecules absorb the UV radiation of such wave lengths as are responsible for the formation of erythema (sunburn) (primarily between 295 and 323 nm with a maximum at 308 nm).

For that reason, all previous sunscreen preparations must be seen as mere “sunburn diminishers” that only guard against or prevent the dramatic effect of skin damage easily evident to the human eye. However, it has already been reported in literature that the even more dangerous damage to the cell caused well before the development of erythema may involve massive DNA damage that may even give rise to the formation of melanoma.

Protection Against Oxidative Stress, Radicals and Environmental Chemicals

In the field of cosmetics a multitude of free radical scavengers and antioxidants has been employed as additives of cosmetic preparations to protect against oxidative stress caused by environmental influences (UV radiation, smoke, chemical substances) as well as by cell-inherent activities. The broad range of agents embraces not only water-soluble free radical scavengers (e.g. ascorbic acid) acting in the liquid inner cell but also fat-soluble antioxidants (e.g. Retionol palmitate or α-tocopherol) that have an effect on the membrane structures. In this context it is rarely mentioned that when these agents are applied topically not more than 3% of them reach deeper skin layers while the rest remains on the skin surface without having any effect.

Co-Solvents

Offering protection against harmful environmental influences human skin consists of several layers (stratum corneum, subdermis, dermis, epidermis) forming a barrier practically impenetrable to foreign substances. Potential routes of penetration open to cosmetic agents are intercellular (only for small polar and non-polar substances by lateral diffusion through the highly organized intercellular lipid layers), intracellular or transfollicular (liposome technology). There is evidence, however, that even when employing the innovative liposome encapsulation in nano-particles cosmetic agents are only capable of penetrating the upper skin layers at an extremely low percentage (<3%) to reach their targeted location where they are intended to take beneficial effect. Larger molecules such as enzymes promising in terms of potential effectiveness (e.g sericin, kollytin, glutathion-S-transferase or the groups of cytokines) can be introduced into living cells to a minute extent only due to steric hindrance, even by applying the most modern methods available.

Compatible Solutes and Hyperthermophiles

Hyperthermophiles are rather extraordinary microorganisms because they grow optimally at temperatures (60-110° C.) that in the event of mesophilic (“normal”) organisms would lead to an extensive damage of cellular structures. In recent years comprehensive research efforts have been made to identify the biochemical components that bring about the remarkable thermal, chemical and physical stabilization of the cell structures. The focus of the research work in this context was on the isolation of thermally stable enzymes due to the fact that numerous enzymatically catalyzed industrial processes are carried out in extreme environments and suitable biocatalysts are required for these applications.

Although many enzymes from hyperthermophilic microorganisms are stable even under elevated temperatures this cannot be generally said of the cellular structures of thermophilic and hyperthermophilic organisms. The high temperature stability of cell structures is—to a remarkable extent—due to low-molecular organic substances (compatible solutes, “hypersolutes”) present in the intracellular environment. In recent years various new hypersolutes could be identified in hyperthermophilic microorganisms for the first time. In some cases it could be clearly shown that these compounds effectively contributed to the protection of cellular structures—first of all enzymes—against heat and dryness bitop has developed technological solutions for the production of solutes from thermophilic microorganisms.

Problems Faced

As interface and surface of the human body the skin is exposed to numerous external stresses. The human skin is an organ that consists of different, specialized cell types—the keratinozytes, melanozytes, Langerhans cells, Merkel cells as well as embedded sensory cells—and protects the body against external influences. In this context a distinction must be made between external physical, chemical and biological factors affecting the human skin. External physical influences are, inter alia, thermal and mechanical influences as well as the effects of radiation such as, for example, UV, VIS and IR radiation. External chemical influences particularly involve, inter alia, the effects of toxins, allergens and substances that attach to desoxyribonucleic acid. External biological influences mean the effects caused by foreign organisms and their metabolic products.

An excessive sun radiation leads to both acute skin damage such as for example sunburns and chronic changes such as, for example, skin aging or skin cancer. Sunburns (erythema solare) primarily develop when the skin is exposed to UV-B radiation. On the other hand, UV-A radiation by comparison has only a minor effect on the occurrence of sunburns. Sunburns of varying degree may develop ranging from a slight reddening to severe burns with blisters. Since these consequences will occur 4-6 h after exposure to radiation at the earliest, it will be too late to take countermeasures. Several sunburns—especially suffered during childhood—will increase the risk of skin cancer significantly. This is due to damage caused particularly to the nucleic acids of human skin cells and a faulty repair of the damaged desoxyribonucleic acid within the nucleus as well as possibly the immunosuppressive effect of UV radiation, i.e. a diminishing of the immune reaction as a result of the exposure to UV rays. An excessive exposure to UV-A and UV-B rays will increase the risk of a premature aging or light aging of the skin, for example in the form of structural changes of the connective tissue (actinic elastosis). An excessive exposure to UV-B rays is viewed as the main cause of chronic skin changes.

Due to changed recreational activities as, for instance, prolonged sunbathing or long-distance holidays in countries where the sun radiation is strong, dangers associated with skin cell damage due to UV exposure have increased severely in recent years which in turn entails a higher skin cancer risk. Moreover, the skin cancer risk has considerably increased lately as a result of the higher UV radiation present on the earth's surface on account of the stratospheric ozone depletion and also the higher live expectancy of mankind.

Aside from the still very topical problem of the lasting damage caused to cells due to UV radiation which by the use of customary sunscreens has hitherto been resolved only inadequately, the long underestimated influence of IR radiation damaging the skin cells thermally (thermal stress denaturizes cell proteins and enzymes) is being the subject of current discussions in research cycles. In the cosmetics industry no agents are known that offer protection against cell stresses caused by elevated temperatures.

Every tissue has an antioxidative potential (AOP) stemming from enzymatic and non-enzymatic antioxidants that in the unstressed cells keep the content of pro-oxidants below a limit that does not mean harm to the healthy cell. In the event these natural antioxidants become deactivated or denaturized the resistance as well as regenerative properties of the skin will decrease significantly.

Hitherto only a few natural substances have been known in preventive cosmetics and dermatology that actively promote the cell-inherent repair mechanisms of the cell and in this manner safeguard the cell “from within” (in vivo) against, for example, elevated UV radiation, IR radiation, heat, free radicals, cold, dryness, osmotic stress or chemical stress. Therefore, the objective was to provide cosmetic and dermatological formulations whose application eliminates or at least alleviates the above described skin problems and which, in particular, are suited to protect and stabilize human skin cells and their organic components.

It is thus a general problem of cosmetics and dermatology that there are no clearly chemically defined and harmless agents and additives of natural origin available to satisfactorily resolve the above mentioned important objectives within the fields of application indicated. The present invention proposes a solution of this problem.

Invention/Beneficial Effects of the Invention

Extremophilic microorganisms (thermophiles and hyperthermophiles) protect themselves against thermal stress by the formation of cyclic diphosphoglycerate. In the presence of these substances these microbial living organisms are capable of existing under extreme environmental conditions because cyclic diphosphoglycerate serves to stabilize and protect the metabolism and the essential organic components against damage.

Surprisingly, it has now been ascertained that cyclic diphosphoglycerate also exhibits its stabilizing effects when applied to the human skin and thus must be seen as ideal agents and additive for cosmetic and dermatological preparations. Cyclic diphosphoglycerate thus represents a universally applicable and completely novel protective principle in the field of cosmetics and dermatology. Therefore, according to the invention the above mentioned problem can be resolved by making use of one or more chemical compounds selected from the compounds of formula Ia (cyclic disphosphoglycerate) in cosmetic and dermatological formulations. The invention relates to the use of one or more compounds selected from the compounds of formula Ia (cyclic diphosphoglycerate),

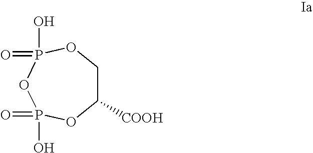

the physiologically compatible salts of compound Ia, the stereoisomeric forms of the compounds of formula Ia as well as of derivatives for the production of a cosmetic or dermatological formulation aimed at the protection and stabilization of human skin cells and their organic constituents such as, for example, proteins, enzymes, membranes, nucleic acids or antioxidants.

As salts the alkaline and alkaline-earth salts are suited, but in particular the Na and K salt. As esters the esters of physiologically compatible alcohols can be employed, in particular of C₁-C₄-alcohols such as methanol, ethanol, propanol or butanol.

The invention relates, in particular, to the use of one or more compounds selected from the compounds of formula Ia, the physiologically compatible salts of the compounds of formula Ia and the stereoisomeric forms of the compounds of formula Ia for the production of a cosmetic or dermatological formulation aimed at the protection and stabilization of human skin-cells and their organic constituents such as, for example, proteins, enzymes, membranes, nucleic acids, antioxidants etc. to guard against physical, chemical and biological influences such as radiation (UV, IR, VIS rays), denaturizing substances, temperature or cold.

The present invention describes this protective principle for the first time. Possible fields of application are versatile and can only be described by way of examples within the framework of the invention. However, the invention shall not be limited in whatever way by giving or describing such examples.

Membranes constitute the natural barrier of cells and cell organellae at the surrounding medium. These membranes enable a constant inner environment to prevail within the individual cells and cell constituents. Biological membranes are multi-component systems that usually consist of lipids, cholesterol and proteins. The exact composition of individual membranes varies to a greater or lesser extent depending the origin of the membranes. However, lipids and proteins are elementary constituents of membranes.

Lipids can be subdivided into so-called “simple” lipids such as fats and waxes and “complex” lipids. As basic structural elements of membranes complex lipids such as glycerophospholipids (glycerine phosphatides) are most commonly found. The general structure of these membrane constituents is derived from sn-glycerine-3-phosphoric acid (see following figure; R1, R2, R3=H).

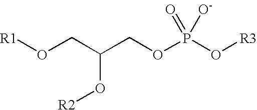

This is phosphoric acid diester in which the glycerine residue usually has condensed with saturated (R1) and unsaturated (R2) fatty acids. The phosphoric acid residue has additionally condensed with a component of alcoholic structure such as inositol or ethanolamine (R3).

Due to its ambivalent structure comprising a hydrophilic phosphate residue and lipophilic fatty acid residues glycerophospholipids are capable of forming aggregates in aqueous solutions (e.g. bilayers or miccelles) in which only the hydrophilic phosphate residue is in contact with the aqueous phase. This structure is also found in the form of closed lamellar vesicles, the so-called liposomes.

An example of the group of lipids with two phospodiester groups, the so-called diphosphatidyl glycerines (cardiolipins), is shown in the following figure:

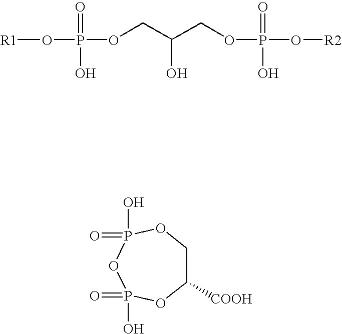

Same as with other compatible solutes from thermophilic or hyperthermophilic microorganisms having a basic lipid structure, structural similarities with diphosphatidyl glycerines can be found in the case of cyclic 2,3 diphosphoglycerates. Within a molecule two phosphate residues are linked with a glycerine residue. In the event of cDPG this is effected by an intramolecular ring closure via the hydroxygroups in 2- and 3-position whereas the diphosphatidyl glycerines are open-chain compounds.

With respect to the stabilizing effect of cDPG it is most important that the substance is present in the form of potassium salt. In connection with the stabilizing effect (thermoadapter) of cDPG the following is discussed:

Binding of hydrophilic protein domains via OH groups (simulation of water), prevention of hydrogen bridging within the protein structure and between proteins during drying and rehydration.

Presentation of a phosphate anion: Stabilization of protein side chains via ionic and van der Waals interaction.

Furthermore, DIP has the following properties:

High chemical and physical stability

pH stability

Light stability

Excellent solubility in water

High biocompatibility

Natural protective substance stemming from R1 microorganisms

No allergenic potential to be expected (substance from extremophiles)

| | Cyclic diphosphoglycerate |
|---|---|
| Molecular weight | 248.02 |
| Empirical formula | C₃H₆O₉P₂ |
| Appearance | solid substance / strongly hygroscopic |
| Half-life (hydrolysis) | T = 120° C.; 150 h |
| CAS number | 88280-54-0 |
| Optimum concentration | 300 mM |

Finally, the solubilizing effect of cDPG substituting the water inside the cell must not be forgotten. Same as the other compatible solutes cDPG is to be seen as the natural solvent of the cell. To actually take effect within the cell biogenic substances have to be solubilized. Hitherto, data on the toxicity of the substance have not been obtained. However, the intracellular concentration of cDPG within the cell (methanothermus fervidus) is above 300 mM so that a high degree of biocompatibility is to be expected here as well!

Temperature Protection of Proteins

It has been shown by experiment that in respect of the thermally induced denaturation of enzymes protective effect of cDPG is excellent and in comparison with a number of other compatible solutes is far and decidedly better. When freezing protein solutions (LDH), cDPG exhibits good stabilizing properties.

Use of the Invention in Trade and Industry

Using the invention in trade and industry encompasses the entire field of cosmetic and dermatological products as well as its application as research reagents in cosmetic and dermatological development work. Moreover, an application in trade and industry also involves the post-operative treatment of the skin (e.g. tending wounds in order to promote healing processes) and the addition of the compounds to plasters, masks and bandages as they are applied to the skin.

Further conceivable applications of compound Ia in cosmetic and dermatological fields are, for example:

Micro-encapsulation of cosmetic agents and additives

Stabilization of liposomes

Moisture dispensing

Free radical and UV protection

Protection against environmental pollutants and salts

Osmostress protectants

### WAYS TO IMPLEMENT THE INVENTION INCLUDING EXAMPLES

The cosmetic and dermatological formulations are manufactured in that one or several compounds of formula Ia (cyclic diphosphoglycerate), the physiologically compatible salts of compounds of formula Ia, the stereoisomeric forms of the compounds of formula Ia or derivatives, if expedient with the aid of auxiliary and/or carrier substances, are appropriately dealt with so as to bring about a suitable formulation.

The auxiliary and carrier substances stem from the group of carrier agents, preservative agents and other customary auxiliary agents. The compounds of formula Ia (cyclic diphosphoglycerate), the physiologically compatible salts of the compounds of formula Ia and the stereoisomeric forms of the compounds of formula Ia as contained in cosmetic and dermatological formulations are for external application. They may for example be used in the form of solutions, suspensions, emulsions, pastes, ointments, gels, creams, lotions, powder, soaps, surfactant-containing cleaning preparations, oils and sprays. Moreover, customary carrier substances, auxiliary substances of any kind and, if thought expedient, further agents may be added to the formulations. Preferable auxiliary substances stem from the group of preservative agents, antioxidants, stabilizers, solutizers, vitamins, coloring agents and de-odorizers.

Aside from one or several compounds selected from the compounds of formula Ia ointments, pastes, creams and gels may contain the customary carrier substances such as, for example, animal and vegetable fats, waxes, paraffins, starch, traganth, cellulose derivatives, polyethylene glycols, silicones, bentonites, silicic acid, talcum and zinc oxide or mixtures/blends of these substances.

Aside from one or several compounds selected from the compounds of formula Ia powders and sprays may contain in addition to the customary carrier substances the customary propellants, e.g. chlorofluorohydrocarbons, propane/butane or dimethyl ether.

Aside from one or several compounds selected from the compounds of formula Ia solutions and emulsions may additionally contain the customary carrier substances such as solvents, solutizers and emulgators or oils.

Aside from one or several compounds selected from the compounds of formula Ia suspensions may contain additional carrier substances such as, for example, water or ethanol.

Further forms of application are, for instance, soaps, surfactant-containing cleansing agents, face and body oils, lipsticks, lip-care sticks, mascara, eyeliners, eye shadowing, rouge, powder, emulsion and wax make-up, as well as sunscreen, pre-sun and after-sun preparations.

The proportion of the compound of formula Ia (cyclic diphosphoglycerate), the physiologically compatible salts of the compounds Ia and the stereoisomeric forms of the compounds of formula Ia in cosmetic and dermatological formulations preferably amounts to 0.0001 to 50% by weight, especially preferred is 0.001 to 10% by weight based on the entire cosmetic formulation.

The cell-inherent antioxidative potential is promoted by the application of cyclic diphosphoglycerate, for example, through its proven stabilizing effect to prevent thermal protein denaturation and deactivation. In this way, an impairment as a result of free radicals is counteracted. As a result of the proven DNA protection of compatible solutes and their protein stabilizing effect the application of cyclic diphosphoglycerate according to the invention is to be regarded as a novel type of active sun protection (e.g. UV and IR radiation) in cosmetics and dermatology.

The following examples are meant to provide elucidation on the present invention but shall by no means limit its scope.

### Example of an O/W Lotion

According to the invention a cyclic diphosphoglycerate-containing lotion (O/W) is made from the following components:

| | Percentage by |
|---|---|
| Components | Weight (% w/v) |
| Paraffin oil (DAB 9) | 8.00 |
| Isopropyl palmitate | 3.00 |
| Petrofatum | 4.00 |
| Cetylstearyl alcohol | 2.00 |
| PEG 40 castor oil | 0.50 |
| Sodium cetylstearyl sulfate | 0.50 |
| Sodium carbomer | 0.40 |
| Cyclic diphosphoglycerate | 0.50 |
| Glycerine | 3.00 |
| α-tocopherol | 0.20 |
| Octylmethooxycinnamate | 5.00 |
| Butylmethoxydibenzoylmethane | 1.00 |
| Preservative agents, coloring matter, perfume | q.s. |
| Water | Ad 100.00 |

As preservative agent 0.05% propylhydroxybenzoate or 0.15% methyl-4-hydroxybenzoate may be used.

### Example of an O/W Cream

According to the invention a cyclic diphosphoglycerate-containing cream (O/W) is made from the following components:

| | Percentage by |
|---|---|
| Components | Weight (% w/v) |
| Paraffin oil (DAB 9) | 7.00 |
| Avocado oil | 3.00 |
| Glycerylmonostearate | 4.00 |
| Cyclic diphosphoglycerate | 0.50 |
| Titanium dioxide | 1.00 |
| Sodium lactate | 3.00 |
| Glycerine | 3.00 |
| Preservative agents, coloring matter, perfume | q.s. |
| Water | Ad 100.00 |

As preservative agent 0.05% propylhydroxybenzoate or 0.15% methyl-4-hydroxybenzoate may be used.

### Example of a Liposome-Containing Gel

According to the invention a liposome-containing gel that contains cyclic diphosphoglycerate is made from the following components:

| | Percentage by |
|---|---|
| Components | weight (% w/v) |
| Lecithin | 6.00 |
| Shea butter | 3.00 |
| cyclic diphosphoglycerate | 0.50 |
| α-tocopherol | 0.20 |
| Biotin | 0.08 |
| Sodium citrate | 0.50 |
| Glycin | 0.20 |
| Urea | 0.20 |
| Sodium PCA | 0.50 |
| Hydrolized collagen | 2.00 |
| Xanthan gum | 1.40 |
| Sorbitol | 3.00 |
| Preservative agents, coloring matter, perfume | q.s. |
| Water | ad 100.00 |

As preservative agent 0.05% propylhydroxybenzoate or 0.15% methyl-4-hydroxybenzoate may be used.

### Example of a Gel

According to the invention a cyclic diphosphoglycerate-containing gel (O/W) is made from the following components:

| | Percentage by |
|---|---|
| Components | Weight (% w/v) |
| Carbopol | 2.00 |
| Triethanolamine | 3.00 |
| Cyclic diphosphoglycerate | 0.50 |
| α-Tocopherylacetate | 0.20 |
| Polyoxyethylene sorbitan fatty acid ester (Tween 20) | 0.50 |
| Glycerine | 2.00 |
| Sodium PCA | 0.50 |
| Hydrolized collagen | 2.00 |
| Preservative agents, coloring matter, perfume | q.s. |
| Water | ad 100.00 |

As preservative agent 0.05% propylhydroxybenzoate or 0.15% methyl-4-hydroxybenzoate may be used.

### Example of a Sunscreen Emulsion

According to the invention a cyclic diphosphoglycerate-containing sunscreen emulsion is made from the following components:

| | Percentage by |
|---|---|
| Components | Weight (% w/v) |
| Cyclomethicone | 2.00 |
| Cetyldimethicone copolyol | 0.20 |
| PEG 22 dodecyl copolymer | 3.00 |
| Paraffin oil (DAB 9) | 2.00 |
| Caprylic acid/Caprinic acid triglyceride | 5.80 |
| Octylmethoxycinnamate | 5.80 |
| Butyl methoxy dibenzoylmethane | 4.00 |
| α-Tocopherylacetate | 0.50 |
| ZnS04 | 0.70 |
| Na3IIEDTA | 0.30 |
| Cyclic diphosphoglycerate | 0.50 |
| Preservative agents, coloring matter, perfume | q.s. |
| Water | ad 100.00 |

As preservative agent 0.05% propylhydroxybenzoate or 0.15% methyl-4-hydroxybenzoate may be used.

### Example of a Sunscreen Lotion

According to the invention an emulgator-free cyclic diphosphoglycerate-containing sunscreen lotion SPF 30 (O/W) is made from the following components:

| | Percentage by |
|---|---|
| Components | Weight (% w/v) |
| Caprylic acid/Caprinic acid triglyceride | 30.00 |
| Uvinul T150 | 4.0 |
| Eusolex 6300 | 2.00 |
| Neo Heliopan OS | 5.00 |
| Parsol 1789 | 2.00 |
| Eusolex T2000 | 4.00 |
| Aerosil R972 | 2.00 |
| Zinc oxide | 2.5 |
| Natrosol Plus 330CS | 0.5 |
| Glycerine | 10.0 |
| Cyclic diphosphoglycerate | 0.50 |
| Preservative agents, coloring matter, perfume | q.s. |
| Water | ad 100.00 |

As preservative agent 0.05% propylhydroxybenzoate or 0.15% methyl-4-hydroxybenzoate may be used.

### Example of a Hair Tonic

According to the invention a cyclic diphosphoglycerate-containing hair lotion is made from the following components:

| | Percentage by |
|---|---|
| Components | Weight (% w/v) |
| Ethanol | 40.00 |
| Diisopropyladipate | 0.10 |
| α-Tocopherylacetate | 0.50 |
| Cyclic diphosphoglycerate | 0.50 |
| Preservative agents, coloring matter, perfume | q.s. |
| Water | ad 100.00 |

As preservative agent 0.05% propylhydroxybenzoate or 0.15% methyl-4-hydroxybenzoate may be used.

### Example of a Spray Formulation

According to the invention a cyclic diphosphoglycerate-containing spray formulation is made from the following components:

| | Percentage by |
|---|---|
| Components | Weight (% w/v) |
| Ethanol | 28.2 |
| α-Tocopherylacetate | 0.10 |
| Cyclic diphosphoglycerate | 0.50 |
| Preservative agents, coloring matter, perfume | q.s. |
| Propane/butane 25/75 | Ad 100.00 |

As preservative agent 0.05% propylhydroxybenzoate or 0.15% methyl-4-hydroxybenzoate may be used.

1. Cosmetic or dermatological topical formulation, preparation or product containing one of the compounds of formula Ia, the physiologically compatible salts of the compounds of formula Ia, the stereoisomeric forms of the compounds of formula Ia and the derivatives of the compounds of formula Ia.

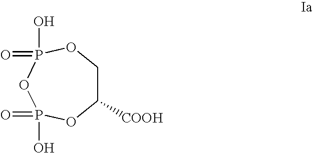

2. Formulation according to claim 1, characterized in that the compounds of formula Ia, the physiologically compatible salts of the compounds of formula Ia, the stereoisomeric forms of the compounds of formula Ia and the derivatives of the compounds of formula Ia are present in concentrations of 0.01 to 50% by weight, preferably 0.05 to 10% by weight, in particular 0.1 to 5% by weight based on the total weight of the formulation, preparation or product.

3. Formulations according to claims 1 or 2 for the protection and stabilization of human skin cells.

4. Formulation according to claims 1 or 2, characterized in that the nucleic acids of human skin cells are stabilized and protected against physical, chemical and biological influences.

5. Formulation according to claims 1 or 2, characterized in that the nucleic acids of human skin cells are protected against UV radiation and IR radiation.

6. Formulation according to claims 1 or 2, characterized in that the nucleic acids of human skin cells are stabilized and protected against denaturizing substances.

7. Formulation according to claims 1 or 2, characterized in that the nucleic acids of human skin cells are stabilized and/or protected against enzymes.

8. Formulation according to claims 1 or 2, characterized in that the human skin cells are stabilized and protected against viruses, viroids and prions.

9. Formulation according to claims 1 or 2, characterized in that human skin cells are stabilized and protected against low-infective agents.

10. Formulation according to claims 1 or 2 for the tending and/or prophylaxis of dry and/or scaly skin.

11. Formulation according to claims 1 or 2 for the protection of the human skin against dryness and/or elevated salt concentrations (higher than 1% w/w).

12. Formulation according to claims 1 or 2 for the protection of cells, proteins, protein complexes, lipoproteins, ribosomes and/or biomembranes of the human skin.

13. Formulation according to claims 1 or 2 for the protection of the microflora of the human skin.

14. Formulation according to claims 1 or 2 for the stabilization of the natural skin barrier.

15. Formulation according to claims 1 or 2 as water-retaining (water-binding) substance.

16. Formulation according to claims 1 or 2 as free radical scavenger and/or antioxidant as well as its use for the treatment of and/or prophylaxis for skin aging caused by oxidative stress and of inflammatory reactions.

17. Formulation according to claims 1 or 2 for enhancing and accelerating the immune reaction of human skin cells for the protection of cells, proteins and/or biomembranes of human skin exposed to physical, chemical and biological stress.

18. Formulation according to claims 1 or 2, characterized in that the cosmetic or dermatological formulation contains one or several UV filters.

19. Formulation according to claims 1 or 2, characterized in that the cosmetic or dermatological formulation contains one or several substances selected from enzymes, vitamins and vitamin derivatives.

20. Formulation according to claims 1 or 2, characterized in that the nucleic acids of the cells of the epidermis are protected and stabilized.

21. Use of one of the compounds of formula Ia, the physiologically compatible salts of the compounds of formula Ia, the stereoisomeric forms of the compounds of formula Ia and the derivatives of the compounds of formula Ia, possibly together with one or several additional agents and additives in a formulation for the external application in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair lotion, a plaster, a bandage or spray.

22. Cosmetic formulation containing one or several compounds selected from the compounds of formula Ia, the physiologically compatible salts of the compounds of formula Ia, the stereoisomeric forms of the compounds of formula Ia and the derivatives of the compounds.

23. Dermatological formulation containing one or several compounds selected from the compounds of formula Ia, the physiologically compatible salts of the compounds of formula Ia, the stereoisomeric forms of the compounds of formula Ia and the derivatives of the compounds.

24. Cosmetic formulation according to claim 22, characterized in that it is provided in the form of a solution, a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair lotion, a plaster, a bandage or spray.

25. Dermatological formulation according to claim 23, characterized in that it is provided in the form of a solution; a suspension, an emulsion, a paste, an ointment, a gel, a cream, a lotion, a powder, a soap, a surfactant-containing cleansing agent, an oil, a lipstick, a lip-care stick, a mascara, an eyeliners, of eye shadowing, rouge, a powder, emulsion and wax make-up, a sunscreen, pre-sun and after-sun preparation, a hair lotion, a plaster, a bandage or spray.
